(19) United States
(12) Patent Application Publication
(10) Pub. No.: US 2025/0313904 A1
(43) Pub. Date: Oct. 9, 2025

(54) SYSTEM AND METHOD FOR GENE EXPRESSION-BASED MONITORING OF PLANKTON STATUS AND ROLES IN AQUACULTURE ECOSYSTEMS

(71) Applicant: The Hong Kong University of Science and Technology, Hong Kong (CN)

(72) Inventor: Longjun WU, Hong Kong (CN)

(21) Appl. No.: 19/173,834

(22) Filed: Apr. 9, 2025

Related U.S. Application Data
(60) Provisional application No. 63/631,455, filed on Apr. 9, 2024.

(51) Int. Cl. C12Q 1/6888; G16B 25/10; G16B 40/20

(52) U.S. Cl. CPC C12Q 1/6888; G16B 25/10; G16B 40/20; C12Q 2600/158

(57) ABSTRACT

A system includes an aquaculture system, a plankton processing system, a gene expression database, a plankton analysis system, and an output module. The plankton processing system collects and processes plankton samples, extracts and sequences RNA, and performs bioinformatic analysis, including gene annotation, transcriptome assembly, and gene expression quantification, outputting gene expression data to the gene expression database. The plankton analysis system retrieves and analyzes gene expression patterns to assess plankton status and ecological roles. The plankton analysis system includes an AI-based module, which establishes correlations between gene expression and ecosystem conditions, and a rule-based module, which analyzes gene functions using predefined biological rules. The output module generates a report providing insights into plankton health, nutrient cycling, and aquaculture ecosystem conditions.

### CROSS-REFERENCE TO RELEVANT APPLICATIONS

The present application claims priority from a U.S. provisional patent application Ser. No. 63/631,455 filed Apr. 9, 2024, and the disclosure of which are incorporated by reference in their entirety.

### TECHNICAL FIELD

The present invention is related to the field of aquaculture ecosystem monitoring and environmental genomics, in particular to a system and a method for assessing the physiological status and ecological roles of plankton in aquaculture environments using an artificial intelligence (AI) predictive model.

### BACKGROUND

The aquaculture system is an ecosystem; in order to achieve a high yield of aquaculture products such as fish, shrimp, and oysters, the healthy development and stability of the ecosystem are very important.

Plankton, including zooplankton and phytoplankton, play important roles in the ecosystem. They maintain the biochemical cycle of the ecosystem and serve as primary producers or major food sources. Their physiological status strongly impacts their functions in the aquaculture ecosystem. The overgrowth of plankton, or the rapid proliferation of certain plankton species, can introduce serious issues into the ecosystem and lead to its collapse. The condition of plankton reflects real-time and ongoing ecological processes.

Therefore, the physiological status of plankton is an important indicator of the current and future condition of the aquaculture ecosystem, and monitoring their status is highly beneficial for aquaculture productivity.

However, in existing approaches, aquaculture ponds or cages only monitor the species composition of plankton, including which species are present in the ecosystem and their population numbers. The approach provides only limited information about plankton in the ecosystem because their physiological status can vary significantly under different environmental conditions, even if the species composition remains the same.

Existing approaches do not provide real-time information on the health conditions of plankton; for example, whether plankton are stressed, dying, growing, or declining. Similarly, existing approaches do not provide real-time insights into the roles and functions of plankton in the ecosystem. For example, they do not determine whether plankton are injecting certain nutrients into the system or consuming them, nor do they indicate whether their current status serves as a warning of potential ecological issues.

Accordingly, there is a need for a monitoring system that uses gene expression analysis to assess the real-time physiological status and ecological functions of plankton, integrating with an artificial intelligence (AI) predictive model for enhanced ecosystem monitoring.

### SUMMARY OF INVENTION

It is an objective of the present invention to provide a system and a method to solve the aforementioned technical problems.

In the present invention, a system and a method are provided to monitor the environmental and health conditions of aquaculture ponds or cages by analyzing the gene expression levels of plankton within the ecosystem. Through transcriptomic sequencing, the physiological status of plankton and their roles in nutrient cycling are assessed by quantifying mRNA transcripts at a given time. The gene expression data offers comprehensive insights into gene activity, and through bioinformatic analysis, combined with known gene functions, the system and the method provide real-time information on the functional roles of plankton within the aquaculture ecosystem.

In accordance with a first aspect of the present invention, a gene expression-based monitoring system for assessing conditions of an aquaculture ecosystem is provided. The system includes an aquaculture system, a plankton processing system, a gene expression database, a plankton analysis system, and an output module. The aquaculture system is configured to cultivate aquatic organisms and serve as an aquaculture ecosystem for plankton populations. The plankton processing system is coupled with the aquaculture system and is configured to collect and process plankton samples from the aquaculture system, extract and sequence RNA of the plankton samples, and perform bioinformatic analysis for the plankton samples, which comprises gene annotation, transcriptome assembly, and gene expression quantification. The plankton processing system is further configured to output gene expression data upon the bioinformatic analysis. The gene expression database is configured to store the gene expression data generated by the plankton processing system. The plankton analysis system is configured to retrieve the gene expression data from the gene expression database and analyze gene expression patterns of the gene expression data to assess plankton status and ecological roles of the plankton samples in the aquaculture ecosystem. The plankton analysis system includes an AI-based plankton analysis module and a rule-based plankton analysis module. The AI-based plankton analysis module is configured to establish correlations between the gene expression patterns of the plankton samples and aquaculture ecosystem conditions through a trained AI model for analysis. The rule-based plankton analysis module is configured to analyze gene functions and metabolic pathways of the plankton samples using predefined biological rules and bioinformatics tools. The output module is configured to generate a report based on at least one analysis result from the plankton analysis system. The report provides insights into plankton health, nutrient cycling, and conditions of the aquaculture ecosystem.

In accordance with a second aspect of the present invention, a gene expression-based monitoring method for assessing conditions of an aquaculture ecosystem is provided. The method include steps as follows: cultivating aquatic organisms in an aquaculture system to create an aquaculture ecosystem for plankton populations; collecting and processing, by a plankton processing system, plankton samples from the aquaculture system; extracting and sequencing RNA of the plankton samples by the plankton processing system; performing, by the plankton processing system, bioinformatic analysis for the plankton samples, comprising gene annotation, transcriptome assembly, and gene expression quantification; outputting gene expression data by the plankton processing system upon the bioinformatic analysis; storing the gene expression data by a gene expression database; retrieving the gene expression data, by a plankton analysis system, from the gene expression database; analyzing, by the plankton analysis system, gene expression patterns of the gene expression data to assess plankton status and ecological roles of the plankton samples in the aquaculture ecosystem. The plankton analysis system includes an AI-based plankton analysis module and a rule-based plankton analysis module. The AI-based plankton analysis module is configured to establish correlations between the gene expression patterns of the plankton samples and aquaculture ecosystem conditions through a trained AI model for analysis. The rule-based plankton analysis module is configured to analyze gene functions and metabolic pathways of the plankton samples using predefined biological rules and bioinformatics tools. The method further includes a step: generating, by an output module, a report based on at least one analysis result from the plankton analysis system, in which the report provides insights into plankton health, nutrient cycling, and conditions of the aquaculture ecosystem.

By this configuration, the gene expression levels of plankton are determined through methods such as mRNA sequencing (or transcriptome sequencing). The gene expression levels are then analyzed based on gene functions and the species they belong to. The plankton's physiological status and ongoing role in nutrient cycling in the ecosystem are assessed through bioinformatic analysis. These data are collected, stored, and compared to different states of the aquaculture system, allowing the system to determine the relationship between plankton gene expression and the real-time condition of the aquaculture ecosystem, as well as its future outlook. The correlation between plankton gene expression and the health status of the aquaculture system is derived through data analysis using AI-based approaches.

### BRIEF DESCRIPTION OF DRAWINGS

Embodiments of the invention are described in more details hereinafter with reference to the drawings, in which:

FIG. 1 illustrates a schematic diagram of an architecture of a gene expression-based monitoring system according to some embodiments of the present invention;

FIG. 2 shows a schematic diagram of a process for assessing a state and condition of an aquaculture ecosystem according to some embodiments of the present invention; and

FIG. 3 illustrates a schematic diagram of an architecture of a gene expression-based monitoring system according to some embodiments of the present invention.

### DETAILED DESCRIPTION OF THE INVENTION

In the following description, systems and methods for gene expression-based monitoring of plankton status and roles in aquaculture ecosystems and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Plankton are a key component of the aquaculture system and serve as an important indicator of its health. Previous research has primarily focused on identifying the species composition of plankton in aquaculture; however, organisms are constantly changing. Even with the same species composition and population size, their physiological conditions can vary significantly. Gene expression levels provide a more precise measure of their current status.

Briefly, the present invention provides systems for monitoring the condition and status of plankton in aquaculture environments by analyzing their gene expression levels, determined through methods such as mRNA sequencing (e.g., transcriptome sequencing). The process includes plankton collection, RNA extraction, sequencing library construction, sequencing, and bioinformatic analysis, which involves gene annotation, species assignment, functional characterization, and pathway analysis. Furthermore, an artificial intelligence (AI) predictive model is integrated into the process for real-time monitoring and analysis.

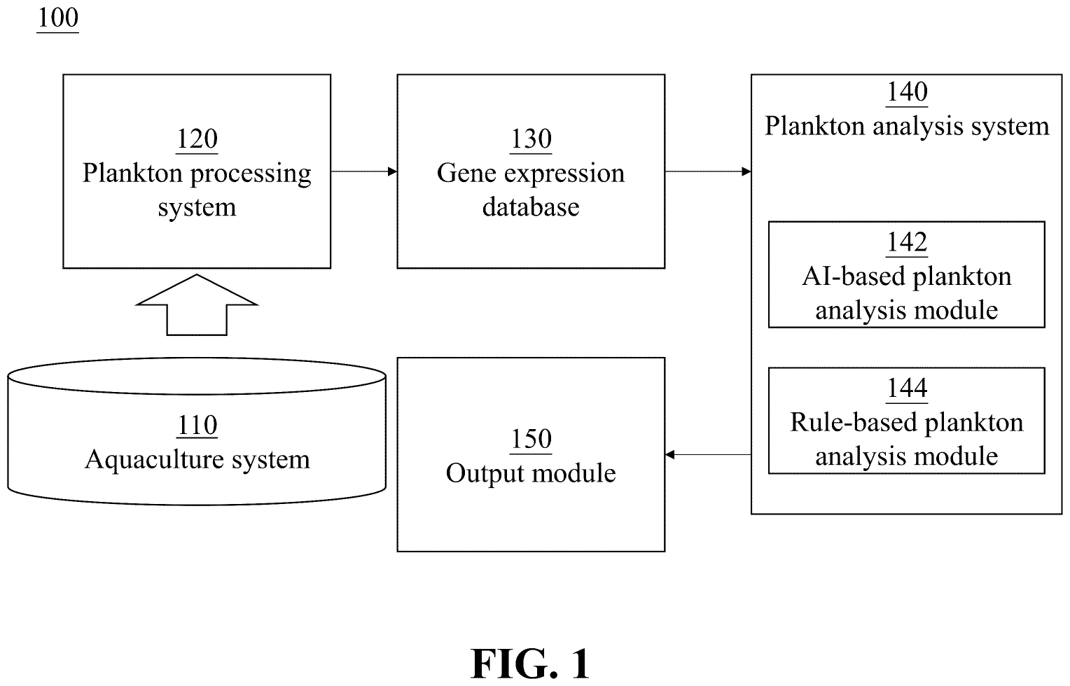

FIG. 1 illustrates a schematic diagram of an architecture of a gene expression-based monitoring system 100 according to some embodiments of the present invention. The gene expression-based monitoring system 100 is applied to real-time data capture for pond or cage aquaculture, including fish, shrimp, and oysters and configured to assesses plankton status and ecological roles in aquaculture ecosystems. Real-time insights into plankton's physiological status provide information on nutrient cycling and the overall health of the aquaculture system. Accordingly, the gene expression-based monitoring system 100 contributes to a more stable and healthier aquaculture environment, leading to higher productivity in fish, shrimp, and oyster farming.

The gene expression-based monitoring system 100 includes an aquaculture system 110, a plankton processing system 120, a gene expression database 130, a plankton analysis system 140, and an output module 150.

The aquaculture system 110 is configured to provide an environment for cultivating aquatic organisms, such as fish, shrimp, oysters, and other marine or freshwater species. In various embodiments, the aquaculture system 110 ranges from small-scale ponds to large, managed aquatic ecosystems designed for commercial production or research.

The plankton processing system 120 is coupled with the aquaculture system 110 for collecting and processing plankton. The plankton processing system 120 is configured to extract, process, and sequence RNA from plankton collected in the aquaculture system 110. Moreover, the plankton processing system 120 can perform transcriptome assembly, genome assembly, gene annotation, mapping of sequencing reads to the assembly, and gene expression quantification.

The gene expression database 130 is configured to store gene expression data generated by the plankton processing system 120, including transcript counts and annotated gene functions. Accordingly, the gene expression database 130 serves as a centralized repository for storing the processed output data from the plankton processing system 120, facilitating downstream analysis.

The plankton analysis system 140 is configured to retrieve the gene expression data stored in the gene expression database 130 and analyze the gene expression data, thereby assessing plankton status and ecological roles. The plankton analysis system 140 includes an AI-based plankton analysis module 142 and a rule-based plankton analysis module 144, which employs non-AI methods for analysis.

In one embodiment, the AI-based plankton analysis module 142 can leverage the rule-based plankton analysis module 144 to enhance its learning process by incorporating predefined biological rules, knowledge-based annotations, and curated pathway analyses. Through the integration of the AI-based plankton analysis module 142 with the rule-based plankton analysis module 144, the AI-based plankton analysis module 142 develops a well-trained model for plankton classification, ecological role identification, and gene expression pattern recognition. In one embodiment, the rule-based plankton analysis module 144 can provide a validation and optimization framework for the AI-based plankton analysis module 142. By cross-referencing AI-generated outputs with rule-based analytical results, the system 100 enhances the performance of AI models, improving robustness, interpretability, and overall analytical precision.

The output module 150 is configured to generate a report based on the analysis results from the plankton analysis system 140. The output module 150 processes and translates the raw analysis data into a human-friendly format, making the information more accessible and understandable. In one embodiment, the output module 150 adds textual explanations to the report, providing insights into plankton health, nutrient cycling, and overall aquaculture ecosystem conditions for the aquaculture management.

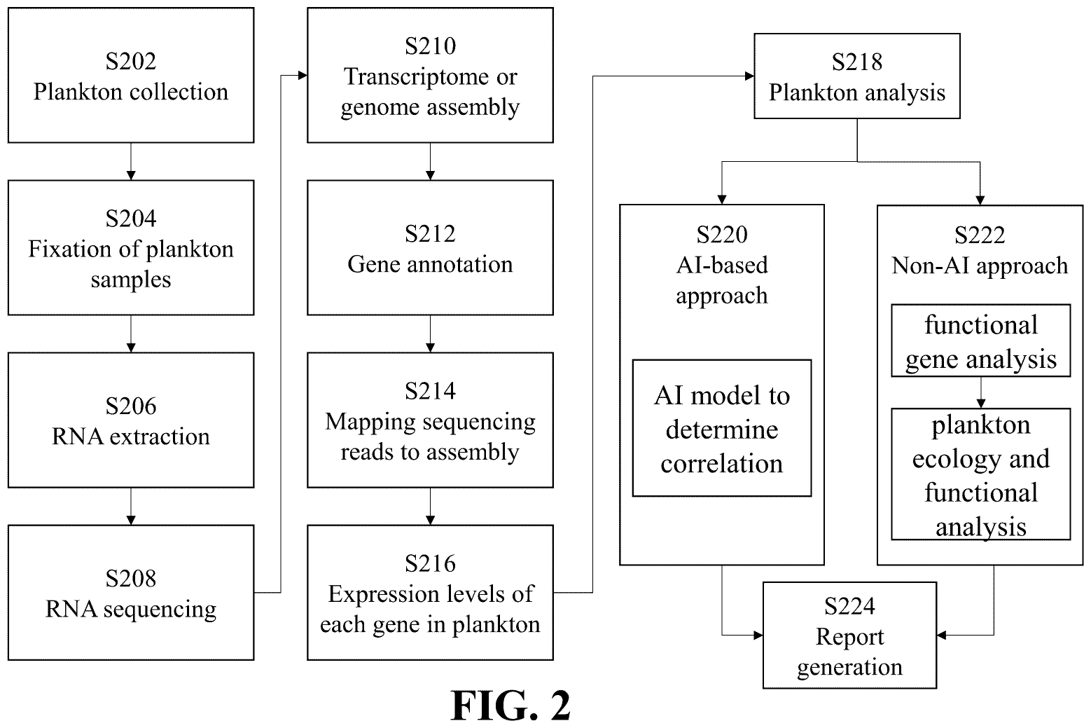

FIG. 2 shows a schematic diagram of a process for assessing a state and condition of an aquaculture ecosystem according to some embodiments of the present invention. As shown in FIG. 1 and FIG. 2, the process is executed by the gene expression-based monitoring system 100 of FIG. 1 and includes steps S202, S204, S206, S208, S210, S212, S214, S216, S218, S220, S222, and S224.

Step S202 involves plankton collection. The plankton processing system 120 collects plankton from the aquaculture system 110 using tools such as a plankton net, pump, or filtration system, depending on the water volume and depth. The collected samples (e.g., plankton) are then transferred to sterile containers to prevent contamination. In some embodiments, for real-time monitoring, the plankton processing system 120 may include an automated plankton sampling device configured to capture periodic samples at different time intervals.

Step S204 involves the fixation of plankton samples. To preserve RNA integrity, plankton samples are fixed using RNA stabilization reagent (e.g., RNAlater) or snap-frozen in liquid nitrogen. In one embodiment, the samples can then be stored at −80° C. until further processing to prevent RNA degradation. In one embodiment, the plankton processing system 120 includes hardware that enables the immediate extraction of RNA without the need for RNA storage.

Step S206 involves RNA extraction. The RNA from the plankton (e.g., the fixed plankton samples obtained from step S204) is then extracted using molecular biology techniques, such as silica-based column extraction kits or phenol-chloroform extraction. In one embodiment, the plankton processing system 120 includes an automated RNA extraction machine for RNA processing.

Step S208 involves RNA sequencing. The extracted RNA from step S206 is sequenced using high-throughput sequencing instruments, such as Illumina sequencers or Oxford Nanopore sequencers. In one embodiment, the plankton processing system 120 includes a sequencing platform, a library preparation system, and a computational resource, cooperated with each other, for RNA sequencing and data processing.

Step S210 involves transcriptome or genome assembly, which is the process of reconstructing full-length transcripts or genomes from the sequenced reads obtained in step S208 (i.e., RNA sequencing). This step is made for identifying gene structures and understanding the functional composition of plankton communities. The sequenced reads are assembled into a transcriptome (if reconstructing expressed genes) or a genome (if assembling full genetic material). Since plankton communities often contain multiple species, the assembly process must account for multi-species datasets, leading to the generation of distinct assemblies for different species. In one embodiment, the plankton processing system 120 includes a high-performance computing (HPC) resource, a reference-based assembly software, and a bioinformatics pipeline for transcriptome or genome assembly in this step.

Step S212 involves gene annotation. Once the transcriptome or genome has been assembled (e.g., in step S210), each gene's name and function are determined through bioinformatic analysis, such as a BLAST-based approach. In some embodiments, to further refine gene annotation, gene sequences are categorized based on their biological roles, metabolic pathways, or taxonomic classification. In some embodiments, the plankton processing system 120 includes a sequence search tool to compare sequences against general genetic reference databases. In some embodiments, gene classification is conducted by the plankton processing system 120 using functional gene annotation databases to group genes into relevant biological functions and pathways.

Step S214 involves mapping sequencing reads to the assembly, which aligns the sequencing reads obtained from Step S208 (i.e., RNA sequencing) with the assembled transcriptome or genome from Step S210 (i.e., transcriptome or genome assembly). Step S212 (i.e., gene annotation) identifies and classifies genes within the assembled transcriptome/genome, providing a reference framework. The step S214 builds upon this reference framework by mapping raw sequencing reads to these annotated genes, enabling the quantification of gene expression levels and facilitating downstream functional analysis. In one embodiment, the plankton processing system 120 includes bioinformatics tools and computational resources for read mapping, utilizing algorithms such as reference-based alignment or mapping techniques to achieve high-precision gene expression analysis.

Step S216 involves gene expression quantification, where the expression level of each gene in the plankton community is determined based on the number of mapped reads (i.e., read counts) obtained from step S214 (i.e., mapping sequencing reads to the assembly). The resulting gene expression data is compiled into a structured dataset and stored in the gene expression database 130, providing a reference for downstream analysis.

After obtaining the expression level of each gene in the plankton community (i.e., step S216), the process moves to step S218, involving plankton analysis. There are two alternative analysis approaches, including steps S220 and S222. The step S220 involves an AI-based approach executed by the AI-based plankton analysis module 142. The step S222 involves a non-AI Approach executed by the rule-based plankton analysis module 144.

In step S220, regarding the AI-based approach, with a collection of plankton gene expression datasets obtained in step S216 and corresponding ecosystem health records from multiple time points (which may be obtained through measurement and recording), the AI-based plankton analysis module 142 implements an AI algorithm to establish correlations between gene expression patterns and the condition of the aquaculture ecosystem. The present invention is not limited to analyzing gene expression patterns within a single aquaculture system. In some embodiments, the AI model/algorithm can incorporate records from other aquaculture systems employing the same or different device/configuration, such as data from another aquaculture pond that cultures the same species. By leveraging data from multiple similar systems/ecosystems, the AI model/algorithm establishes correlations between gene expression patterns and the condition of various aquaculture ecosystems, allowing for broader applicability and transferability across different but comparable environments.

The AI-based plankton analysis module 142 includes a trained AI model, which has been developed using historical gene expression data and ecosystem health labels. This configuration allows the AI model to recognize patterns in gene expression that correlate with ecosystem stability or distress. For example, the AI model identifies specific genes, species, or taxonomic groups that serve as strong indicators of ecosystem health. Once the model is sufficiently trained, it can predict the ecosystem's health condition based solely on real-time plankton gene expression profiles.

For example, the development of the trained AI model relies on a dataset composed of historical gene expression data from plankton samples and ecosystem health labels that classify past environmental conditions. The labels are derived from measured environmental parameters (e.g., dissolved oxygen levels, temperature, salinity, and nutrient concentrations) and biological indicators (e.g., fish mortality rates, algal bloom occurrences, or shifts in plankton community composition). In some embodiments, in a dataset where past records show low dissolved oxygen levels corresponding with high expression of anaerobic metabolism genes in plankton, the AI model of the AI-based plankton analysis module 142 learns to associate similar gene expression patterns with potential hypoxia events in real-time monitoring. Similarly, if historical data links elevated expression of nitrogen assimilation genes with nitrate depletion events, the AI model of the AI-based plankton analysis module 142 can predict nutrient imbalances before they escalate. By training on diverse datasets spanning seasonal cycles, pollution events, and aquaculture disruptions, the AI-based plankton analysis module 142 improves its ability to detect early warning signs of ecosystem distress, enabling proactive management interventions.

In some embodiments, during training or analysis, the AI model can incorporate additional input sources, integrating environmental parameters, health conditions of aquaculture species, or a combination thereof. Environmental parameters may include temperature, oxygen levels, salinity, nitrate, and phosphate concentrations, which directly influence ecosystem conditions. The health conditions of aquaculture species, such as fish and shrimp, may include growth rate, locomotion speed, feeding rate, and gene expression markers, providing an indirect measure of ecosystem health. These additional inputs enhance the AI model's ability to make more accurate predictions about ecosystem stress, stability, or nutrient imbalances.

The rule-based plankton analysis module 144 executes two processes: functional gene analysis and plankton ecology and functional analysis.

In the functional gene analysis process, the rule-based plankton analysis module 144 identifies gene functions and metabolic pathways using established bioinformatics tools. For example, Gene Ontology (GO) classifies genes based on their biological processes, molecular functions, and cellular components. For example, the Kyoto Encyclopedia of Genes and Genomes (KEGG) maps genes to known metabolic and signaling pathways. These analyses help determine how plankton contribute to nutrient cycling, stress responses, and ecosystem regulation. The rule-based plankton analysis module 144 also records which gene functions or pathways are enriched based on expression intensity, revealing biochemical trends that may indicate environmental stress or metabolic adaptations.

Building on this, in the plankton ecology and functional analysis process, the rule-based plankton analysis module 144 interprets gene expression data to assess the overall ecosystem condition. Based on the expression intensity of specific gene functions or pathways, conclusions about the aquaculture ecosystem's state can be drawn. Example 1: If stress-related genes are highly expressed, the rule-based plankton analysis module 144 indicates that the ecosystem is experiencing environmental stress (e.g., temperature fluctuations, pollution). Example 2: If nitrogen biosynthesis-related genes are highly expressed, it means a nitrogen deficiency in the ecosystem, potentially affecting plankton and aquaculture species. By analyzing the expression intensity of specific gene functions or pathways, the rule-based plankton analysis module 144 determines key ecological indicators, including nutrient availability, water quality shifts, and environmental stressors.

Finally, in step S224, the state and the condition of the aquaculture ecosystem are output through the output module 150, generating a comprehensive report based on the validated analysis results. The output report consolidates insights from the AI-based plankton analysis module 142, the rule-based plankton analysis module 144, or both.

The output report may include ecological indicators, such as: (1) ecosystem health status: normal, moderate stress, or critical condition; (2) findings: detection of environmental stressors, such as low oxygen levels, high salinity, or nutrient deficiencies; (3) plankton gene expression trends: identification of highly expressed stress-related genes, nitrogen metabolism genes, or photosynthesis-related genes; (4) AI vs. non-AI verification results: confirmation of AI-predicted anomalies or identification of discrepancies requiring further investigation.

The analysis of the system can be performed at the single-species or multi-species level (such as meta-transcriptomics). The correlation between gene expression patterns and the health of the aquaculture ecosystem can be derived from further analysis of the collected gene expression data and historical ecosystem records. Additionally, AI-based methods can be employed to enhance predictive modeling and data interpretation.

Furthermore, the implementation of the AI-based and non-AI approaches can be selected in different scenarios. The selection between the AI-based approach and the functional gene analysis approach depends on the available data, analytical goals, and real-time monitoring needs.

In one embodiment, in well-established aquaculture facilities where large-scale historical gene expression datasets and environmental records are available, the AI-based approach can be implemented to develop an automated monitoring and prediction system. By training the AI model of the AI-based plankton analysis module 142 on past data, the AI-based plankton analysis module 142 can continuously assess the ecosystem's health, identify potential risks (such as stress conditions or nutrient imbalances), and provide real-time alerts to aquaculture operators. This way is particularly useful for large-scale commercial farms where rapid decision-making is required to prevent adverse environmental effects.

In one embodiment, in small-scale aquaculture setups or research environments where AI training data is insufficient or unavailable, the non-AI approach can be applied. The rule-based plankton analysis module 144 executes functional gene analysis using GO and KEGG, allowing researchers to manually interpret how specific genes respond to environmental changes. For example, if a research team is investigating the effects of temperature fluctuations on plankton, the functional gene analysis method by the rule-based plankton analysis module 144 can reveal which stress-related pathways are upregulated, providing direct insights without requiring extensive prior datasets.

In one embodiment, in scenarios where both predictive monitoring and detailed mechanistic understanding are needed, a hybrid approach combining AI and functional gene analysis can be applied. The AI-based plankton analysis module 142 can rapidly process large-scale expression data and predict ecosystem changes, and the rule-based plankton analysis module 144 executes GO/KEGG analysis to validate and interpret the biological significance of the AI findings.

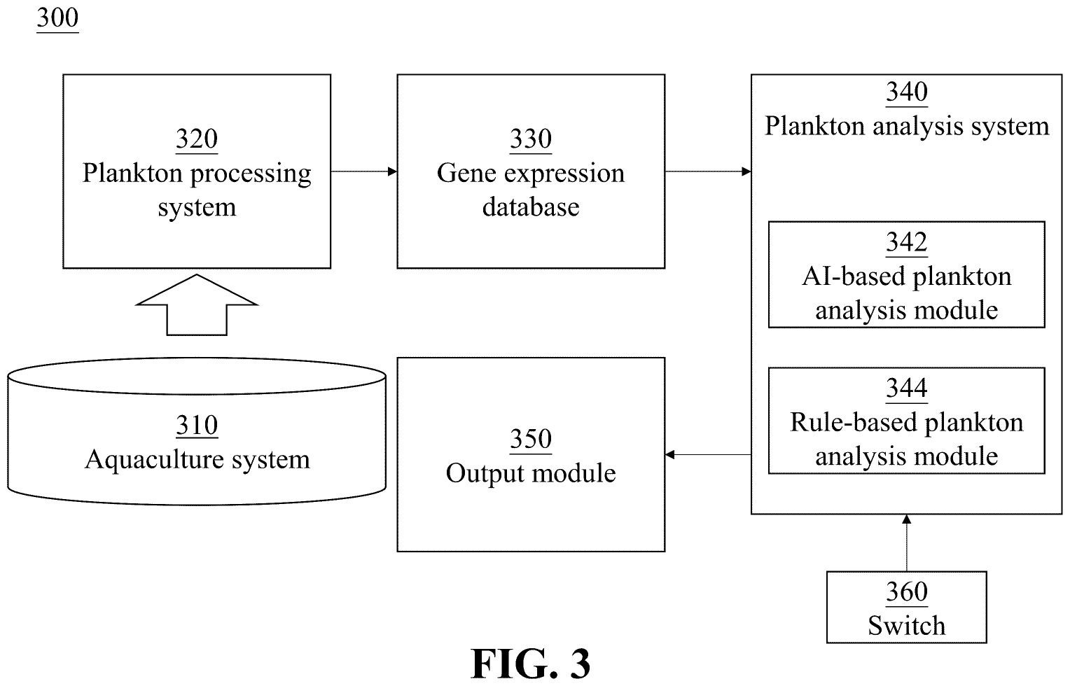

FIG. 3 illustrates a schematic diagram of an architecture of a gene expression-based monitoring system 300 according to some embodiments of the present invention. The configuration of the gene expression-based monitoring system 300 is similar to that of the gene expression-based monitoring system 100, except that the gene expression-based monitoring system 300 further includes a switch 360.

To enhance the reliability of ecosystem assessments, the system 300 incorporates a switch mechanism using the switch 360 that activates rule-based plankton analysis module 344 when the AI-based plankton analysis module 342 detects high-intensity ecological anomalies. The switch mechanism enables self-verification by cross-validating AI predictions with functional gene analysis.

Specifically, when the AI-based plankton analysis module 342 identifies an ecosystem condition that significantly deviates from normal parameters or predefined ecological thresholds (e.g., extreme environmental stress, abnormal nutrient cycling, or unexpected shifts in gene expression patterns), the rule-based plankton analysis module 344 is triggered to perform a further secondary assessment using the non-AI approach, which involves functional gene analysis and pathway-based interpretation in details. Through this validation process, the system 300 independently verifies whether the non-AI analysis corroborates the AI-predicted anomaly. If both approaches indicate the same ecological condition (e.g., stress-related gene enrichment in both analyses), the system 300 confirms the AI-detected anomaly as accurate. Conversely, if discrepancies arise, further analysis may be required to refine the AI model or identify potential misclassifications.

The functional units and modules of the apparatuses and methods in accordance with the embodiments disclosed herein may be implemented using computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes executing in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can be included, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

What is claimed is:

1. A gene expression-based monitoring system for assessing conditions of an aquaculture ecosystem, comprising:
an aquaculture system configured to cultivate aquatic organisms and serve as an aquaculture ecosystem for plankton populations;
a plankton processing system coupled with the aquaculture system and configured to collect and process plankton samples from the aquaculture system, extract and sequence RNA of the plankton samples, and perform bioinformatic analysis for the plankton samples, which comprises gene annotation, transcriptome assembly, and gene expression quantification, wherein the plankton processing system is further configured to output gene expression data upon the bioinformatic analysis;
a gene expression database configured to store the gene expression data generated by the plankton processing system;
a plankton analysis system configured to retrieve the gene expression data from the gene expression database and analyze gene expression patterns of the gene expression data to assess plankton status and ecological roles of the plankton samples in the aquaculture ecosystem, wherein the plankton analysis system comprises:
an AI-based plankton analysis module configured to establish correlations between the gene expression patterns of the plankton samples and aquaculture ecosystem conditions through a trained AI model for analysis; and
a rule-based plankton analysis module configured to analyze gene functions and metabolic pathways of the plankton samples using predefined biological rules and bioinformatics tools; and
an output module configured to generate a report based on at least one analysis result from the plankton analysis system, wherein the report provides insights into plankton health, nutrient cycling, and conditions of the aquaculture ecosystem.

2. The gene expression-based monitoring system of claim 1, wherein the trained AI model of the AI-based plankton analysis module is trained using historical gene expression data and corresponding ecosystem health records from multiple time points.

3. The gene expression-based monitoring system of claim 2, wherein the AI-based plankton analysis module is further configured to incorporate a first input source into the trained AI model, and wherein the first input source comprises environmental parameters including temperature, oxygen levels, salinity, nitrate, phosphate concentrations, or combinations thereof.

4. The gene expression-based monitoring system of claim 3, wherein the AI-based plankton analysis module is further configured to incorporate a second input source into the trained AI model, and wherein the second input source comprises aquaculture species health indicators including growth rate, locomotion speed, feeding rate, or combinations thereof.

5. The gene expression-based monitoring system of claim 4, wherein the AI-based plankton analysis module is further configured to identify specific genes, species, or taxonomic groups that serve as indicators of an ecosystem health condition through the trained AI model.

6. The gene expression-based monitoring system of claim 5, wherein the AI-based plankton analysis module predicts the ecosystem health condition based solely on real-time plankton gene expression profiles.

7. The gene expression-based monitoring system of claim 1, wherein the rule-based plankton analysis module is further configured to perform a functional gene analysis process using Gene Ontology (GO) classification and Kyoto Encyclopedia of Genes and Genomes (KEGG) pathway mapping to interpret gene expression trends.

8. The gene expression-based monitoring system of claim 1, further comprising:
a switch configured to activate the rule-based plankton analysis module when the AI-based plankton analysis module detects high-intensity ecological anomalies, enabling self-verification of AI-predicted anomalies through cross-validation with functional gene analysis.

9. The gene expression-based monitoring system of claim 8, wherein the switch is triggered when the AI-based plankton analysis module detects a significant deviation from predefined ecological thresholds, including extreme environmental stress, abnormal nutrient cycling, or unexpected shifts in gene expression patterns.

10. A gene expression-based monitoring method for assessing conditions of an aquaculture ecosystem, comprising:
cultivating aquatic organisms in an aquaculture system to create an aquaculture ecosystem for plankton populations;
collecting and processing, by a plankton processing system, plankton samples from the aquaculture system;
extracting and sequencing RNA of the plankton samples by the plankton processing system;
performing, by the plankton processing system, bioinformatic analysis for the plankton samples, comprising gene annotation, transcriptome assembly, and gene expression quantification;
outputting gene expression data by the plankton processing system upon the bioinformatic analysis;
storing the gene expression data by a gene expression database;
retrieving the gene expression data, by a plankton analysis system, from the gene expression database;
analyzing, by the plankton analysis system, gene expression patterns of the gene expression data to assess plankton status and ecological roles of the plankton samples in the aquaculture ecosystem, wherein the plankton analysis system comprises:
an AI-based plankton analysis module configured to establish correlations between the gene expression patterns of the plankton samples and aquaculture ecosystem conditions through a trained AI model for analysis; and
a rule-based plankton analysis module configured to analyze gene functions and metabolic pathways of the plankton samples using predefined biological rules and bioinformatics tools; and
generating, by an output module, a report based on at least one analysis result from the plankton analysis system, wherein the report provides insights into plankton health, nutrient cycling, and conditions of the aquaculture ecosystem.

11. The gene expression-based monitoring method of claim 10, wherein the trained AI model of the AI-based plankton analysis module is trained using historical gene expression data and corresponding ecosystem health records from multiple time points.

12. The gene expression-based monitoring method of claim 11, further comprising:
incorporating a first input source into the trained AI model, wherein the first input source comprises environmental parameters including temperature, oxygen levels, salinity, nitrate, phosphate concentrations, or combinations thereof.

13. The gene expression-based monitoring method of claim 12, further comprising:
incorporating a second input source into the trained AI model, wherein the second input source comprises aquaculture species health indicators including growth rate, locomotion speed, feeding rate, or combinations thereof.

14. The gene expression-based monitoring method of claim 13, further comprising:
identifying specific genes, species, or taxonomic groups that serve as indicators of an ecosystem health condition by the AI-based plankton analysis module.

15. The gene expression-based monitoring method of claim 14, wherein the AI-based plankton analysis module predicts the ecosystem health condition based solely on real-time plankton gene expression profiles.

16. The gene expression-based monitoring method of claim 10, wherein the rule-based plankton analysis module performs a functional gene analysis process using Gene Ontology (GO) classification and Kyoto Encyclopedia of Genes and Genomes (KEGG) pathway mapping to interpret gene expression trends.

17. The gene expression-based monitoring method of claim 10, further comprising:
activating the rule-based plankton analysis module by a switch when the AI-based plankton analysis module detects high-intensity ecological anomalies, enabling self-verification of AI-predicted anomalies through cross-validation with functional gene analysis.

18. The gene expression-based monitoring method of claim 17, wherein the switch is triggered when the AI-based plankton analysis module detects a significant deviation from predefined ecological thresholds, including extreme environmental stress, abnormal nutrient cycling, or unexpected shifts in gene expression patterns.
